(19) United States
(12) Patent Application Publication
(10) Pub. No.: US 2006/0024693 A1
(43) Pub. Date: Feb. 2, 2006

(54) Identification of genes or polypeptides the expression of which correlates to fertility, ovarian function and/or fetal/newborn viability

(75) Inventors: Jose Cibelli, East Lansing, MI (US); Javier Crosby, Santiago (CL); Emilio Fernandez, Santiago (CL); Arif Kocabas, East Lansing, MI (US); Guilherme Jordao De Magalhaes Rosa, East Lansing, MI (US)

Correspondence Address: CROWELL & MORING LLP;INTELLECTUAL PROPERTY GROUP, P.O. BOX 14300, WASHINGTON, DC, 20044-4300, US

(73) Assignee: Michigan State University, East Lansing, MI (US)

(21) Appl. No.: 11/091,883

(22) Filed: Mar. 29, 2005

Related U.S. Application Data
(60) Provisional application No. 60/556,875, filed on Mar. 29, 2004.

(51) Int. Cl. C12Q 1/68; C12N 5/06

(52) U.S. Cl. 435/6

(57) ABSTRACT

A genetic means of determining whether a female subject produces “pregnancy competent” oocytes is provided. The means comprises detecting the level of expression of one or more genes that are expressed at characteristic levels (upregulated or downregulated) in cumulus cells derived from pregnancy competent oocytes. This characteristic gene expression level, or pattern referred to herein as the “pregnancy signature”, also can be used to identify subjects with underlying conditions that impair or prevent the development of a viable pregnancy, e.g., pre-menopausal condition, other hormonal dysfunction, ovarian dysfunction, ovarian cyst, cancer or other cell proliferation disorder, autoimmune disease and the like. Microarrays containing “pregnancy signature” genes or corresponding polypeptides provide another preferred aspect of the invention. Still further, the subject invention can be used to derive animal models, e.g., non-human primate animal models, for the evaluation of the efficacy of putative female fertility treatments.

### CROSS REFERENCE TO RELATED APPLICATIONS

Ths application claims the benefit of provisional application No. 60/556,875 filed Mar. 29, 2004, which is incorporated herein by reference in its entirety.

### FIELD OF THE INVENTION

The present invention provides genetic methods that provide for the identification of “pregnancy competent” oocytes, i.e., oocytes that when fertilized and transferred to a suitable uterine environment are capable of yielding a viable pregnancy. The present invention further provides genetic methods of identifying subjects, preferably human females having impaired fertility function, e.g., as a result of impaired ovarian function, e.g., as a result of age (menopause) or an underlying disease condition.

Also, the invention provides methods of evaluating the efficacy of a putative fertility treatment based on its effect on the expression of specific genes.

### BACKGROUND OF THE INVENTION

Currently, there is no available genetic procedures for identifying whether a female subject produces oocytes that are “pregnancy competent”, i.e., which when fertilized by natural or artificial means are capable of giving rise to embryos that in turn are capable of yielding viable offspring when transferred to an appropriate uterine environment. Rather, conventional fertility assessment methods assess fertility e.g., based on hormonal levels, visual inspection of numbers and quality of oocytes, surgical or non-invasive (MRI) inspection of the female reproduction system organs, and the like. Often, when a woman has a problem in producing a viable pregnancy after a prolonged duration, e.g., more than a year, the diagnosis may be an “unexplained” fertility problem and the woman advised to simply keep trying or to seek other options, e.g., adoption or surrogacy. Therefore, providing alternative and more predictive methods for identifying women with fertility problems would be highly desirable. Likewise, novel and improved methods for treating fertility problems would be highly desirable.

Still further, the identification of women with fertility problems, preferably earlier on than by current methods is desirable, as fertility problems may correlate to other health issues that preclude pregnancy, e.g., cancer, menopausal condition, hormonal dysfunction, ovarian cyst, or other underlying disease or health related problems.

### BRIEF DESCRIPTION AND OBJECTS OF THE INVENTION

It is an object of the invention to provide a novel and improved method of detecting infertility problems and the genetic basis thereof.

It is a more specific object of the invention to provide a novel method of detecting female fertility or infertility which method comprises evaluating the capability of oocytes produced by said female to potentially give rise to a viable pregnancy upon fertilization and transferral into a suitable uterine environment, wherein said method involves detecting the levels of expression of specific (“pregnancy signature”) genes or polypeptides encoded thereby.

It is another specific object of the invention to provide a method of evaluating whether a subject produces oocytes capable of giving rise to a viable pregnancy comprising:
(i) measuring the expression levels of genes in a oocyto-derived cell, e.g., a cumulus cell, wherein said genes are expressed or not expressed at characteristic levels (“pregnancy signature”) in cells associated with oocytes capable of yielding a viable pregnancy; and
(ii) detecting the “pregnancy potential” of said oocytes based on the level of similarity of said gene expression pattern to said “pregnancy signature”.

It is another specific object of the invention to identify a female subject putatively having a condition that inhibits or prevents pregnancy by detecting whether said subject produces oocytes associated with cells, e.g., cumulus cells, which do not express one or more genes in a manner characteristic of “pregnancy competent” oocytes; wherein said method comprises detecting the expression of said one or more “pregnancy signature” genes in at least one cell derived from an oocyte isolated from said female subject; and thereby identifying the subject as potentially having a health problem which prevents or precludes fertility based on an abnormal expression pattern of at least one of said “pregnancy signature” genes.

It is another object of the invention to provide a method of evaluating the efficacy of a female fertility treatment which comprises:
(i) treating a female subject putatively having a problem that prevents or inhibits her from having a “viable pregnancy” and
(ii) isolating at least one oocyte from said female subject after said fertility treatment;
(iii) isolating at least one cell from said isolated oocyte, preferably cumulus cell, and detecting the level of expression of at least one gene that is expressed at a characteristic level of expression in “pregnancy competent” oocytes; and
(iv) determining the putative efficacy of said fertility treatment based on whether said gene is expressed at a level characteristic of “pregnancy competent” oocytes as a result of treatment.

It is another object of the invention to provide animal models for evaluating the efficacy of putative fertility treatments comprising identifying genes which are expressed at characteristic levels in cumulus cells derived from pregnancy competent oocytes of a non-human animal, e.g., a non-human primate; and assessing the efficacy of a putative fertility treatment in said non-human animal based on its effect on said gene expression levels, i.e., whether said treatment results in said gene expression levels better mimicking gene expression levels observed in cumulus cells associated with pregnancy competent oocytes, (“pregnancy signature”).

### BRIEF DESCRIPTION OF THE FIGURES

FIGS. 1A-1C and 1D-1I depict schematically a genetic fertility testing method according to the invention. FIG. 1A shows a freshly ovulated egg containing a polar body, zona pellucida and cumulus cells. FIG. 1B shows the fertilization and transferral of this egg into a uterine environment. FIG. 1C shows the recovery of cumulus cells from the oocytes shown in 1A which are to be used for genetic testing. FIG. 1D-1I show the isolation of RNAs from said cumulus cells, microarray analysis of said RNAs, validation of 100 genes by real time RT-PCR, correlation of the levels of expression of said genes (upregulated or downregulated) to the ability of an oocyte to give rise to a viable pregnancy, and the use of this gene expression profile to identify a set of genes, the expression of which correlates to the capability of an oocyte to yield a viable pregnancy (“pregnancy signature”)

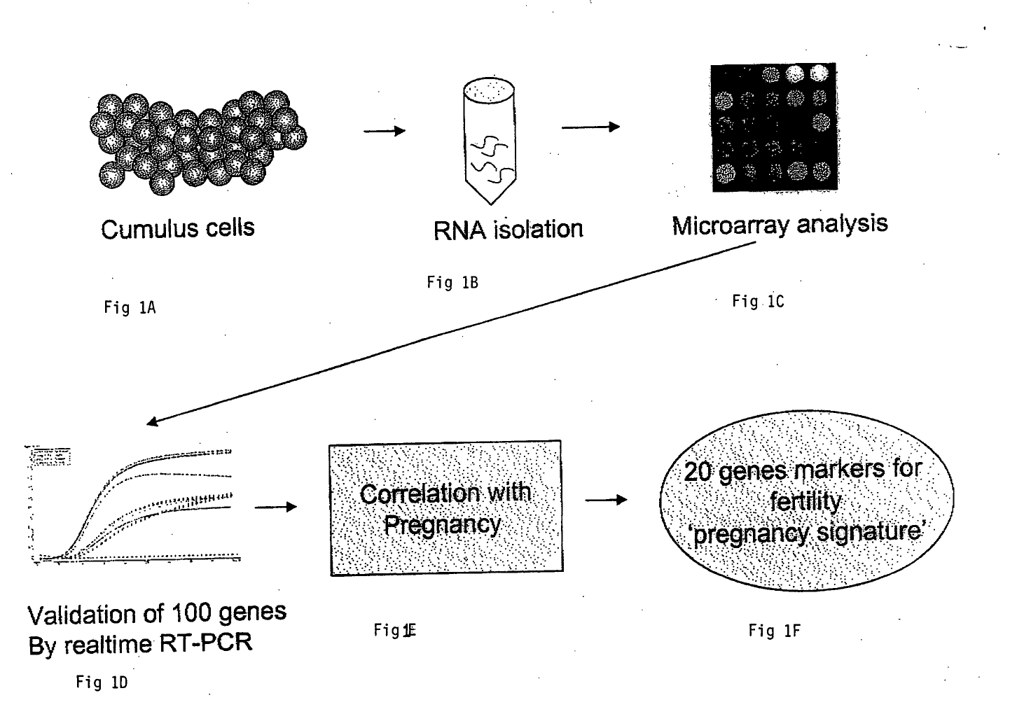

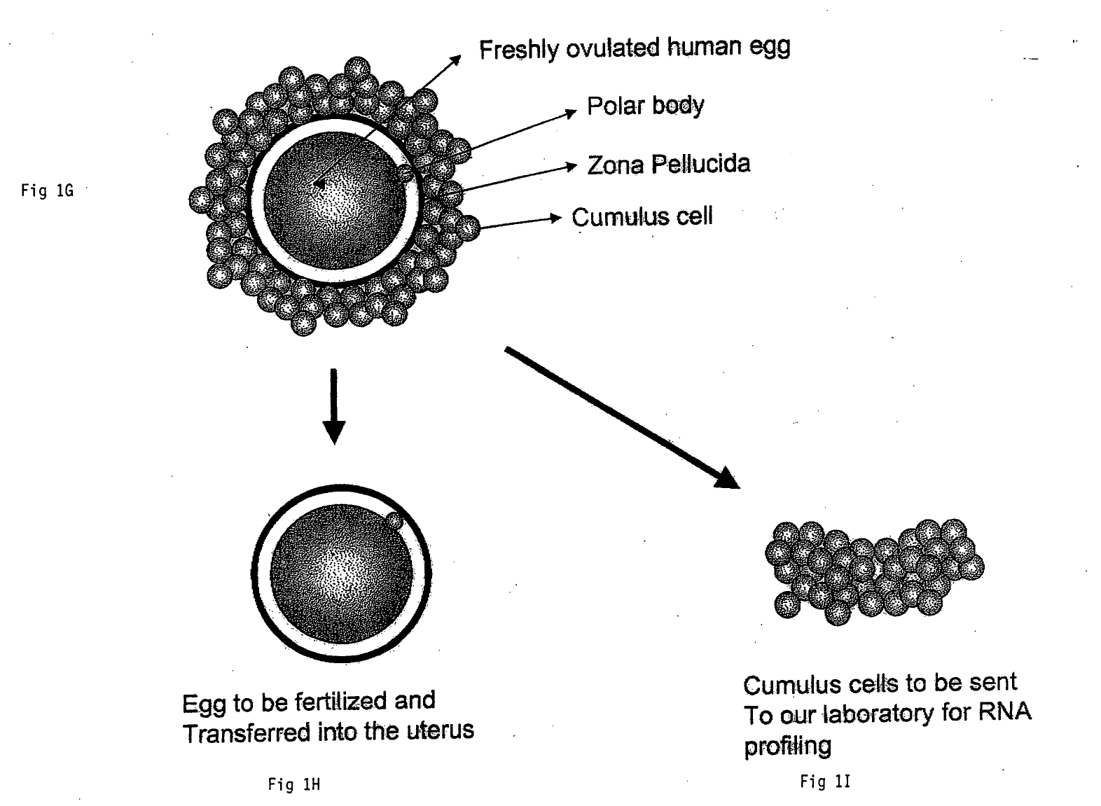

### DETAILED DESCRIPTION OF THE INVENTION

Prior to discussing the invention in more detail, the following definitions are provided. Otherwise all words and phrases in this application are to be construed by their ordinary meaning, as they would be interpreted by an ordinary skilled artisan within the context of the invention.

“Pregnancy-competent oocytes”: refers to a female gamete or egg that when fertilized by natural or artificial means is capable of yielding a viable pregnancy when it is comprised in a suitable uterine environment.

“Viable-pregnancy”: refers to the development of a fertilized oocyte when contained in a suitable uterine environment and its development into a viable fetus, which in turn develops into a viable offspring absent a procedure or event that terminates said pregnancy.

“Cumulus cell” refers to a cell comprised in a mass of cells that surrounds an oocyte. These cells are believed to be involved in providing an oocyte nutritional and or other requirements that are necessary to yield an oocyte which upon fertilization is “pregnancy competent”.

“Differential gene expression” refer to genes the expression of which varies within a tissue of interest; herein preferably a cell from an oocyte, e.g., a cumulus cell.

“Real Time RT-PCR”: refers to a method or device used therein that allows for the simultaneous amplification and quantification of specific RNA transcripts in a sample.

“Microarray analysis”: refers to the quantification of the expression levels of specific genes in a particular sample, e.g., tissue or cell sample.

“Pregnancy signature”: refers to a phrase coined by the inventors which refers to the characteristics levels of expression of a set of one or more genes, preferably at least 5, more preferably at least 10 to 20 genes, and still more preferably, at least 50 to 100 genes, that are expressed at characteristic levels in oocyte cells, preferably cumulus cells, that surround “pregnancy competent” oocytes. This is intended to encompass the level at which the gene is expressed and the distribution of gene expression within cells analyzed.

“Pregnancy signature gene”: refers to a gene which is expressed at characteristic levels by a cell, e.g., cumulus cell, on a “pregnancy competent” oocyte.

“IVF”: refers to in vitro fertilization.

“Zona pellucida” refers to the outermost region of an oocyte.

“Method for detecting differential expressed genes” encompasses any known method for evaluating differential gene expression. Examples include indexing differential display reverse transcription polymorase chain reaction (DDRT-PCR; Mahadeva et al, 1998, J. Mol. Biol. 284:1391-1318; WO 94/01582; subtractive mRNA hybridization (See Advanced Mol. Biol.; R. M. Twyman (1999) Bios Scientific Publishers, Oxford, p. 334, the use of nucleic acid arrays or microarrays (see Nature Genetics, 1999, vol. 21, Suppl. 1061) and the serial analysis of gene expression.

(SAGE Valculesev et al, Science (1995) 270:484-487) and real time PCR (RT-PCR). For example, differential levels of a transcribed gene in an oocyte cell can be detected by use of Northern blotting, and/or RT-PCR.

Preferably, the “pregnancy signature” genes will be detected by hybridization of RNA or DNA to DNA chips, e.g., filter arrays comprising cDNA sequences or glass chips containing cDNA or in situ synthesized oligonucleotide sequences. Filtered arrays are typically better for high and medium abundance genes DNA chips can detect low abundance genes.

Alternatively, polypeptide arrays comprising the polypeptides encoded by pregnancy signature genes or antibodies that bind thereto may be produced and used for diagnosis.

As noted above, the present invention preferably provides a novel method of detecting whether a female subject, human or non-human, produces “pregnancy competent” oocytes. The method involves detecting the levels of expression of one or more genes that are expressed or not expressed at characteristic levels by cumulus cells associated with (surrounding) oocytes that are “pregnancy competent”, i.e., which when fertilized by natural or artificial means (IVF), and transferred into a suitable uterine environment are capable of yielding a viable pregnancy, i.e., embryo that develops into a viable fetus and eventually an offspring unless the pregnancy is terminated by some event or procedure, e.g., a surgical or hormonal intervention.

In preferred embodiments, the invention will be used to identify women subjects who produce or do not produce pregnancy competent oocytes. However, the inventive methods are applicable to non-human animals as well, e.g., other mammals, avians, amphibians, reptiles, et al. For example, the subject invention may be used to derive animal models for the study of putative female fertility treatments.

Additionally, the present invention may be used to identify female subjects who have an abnormality that precludes or inhibits their ability to produce pregnancy competent oocytes, e.g., ovarian dysfunction, ovarian cyst, pre-menopausal or menopausal condition, cancer, autoimmune disorder, hormonal dysfunction, cell proliferation disorder, or another health condition that inhibits or precludes the development of pregnancy competent oocytes.

For example, subjects who do not express specific pregnancy signature genes at characteristic expression levels will be screened to assess whether they have an underlying health condition that precludes them from producing pregnancy competent oocytes. Particularly, such subjects will be screened to assess whether they are exhibiting signs of menopause, whether they have a cancer, autoimmune disease or ovarian abnormality, e.g., ovarian cyst, or whether they have another health condition, e.g., hormonal disorder, allergic disorder, etc., that may preclude the development of “pregnancy competent” oocytes.

Additionally, the subject methods may be used to assess the efficacy of putative female fertility treatments in humans or non-human female subjects. Essentially, such methods will comprise treating a female subject, preferably a woman, with a putative fertility enhancing treatment, isolating at least one oocyte from said woman after treatment, optionally further isolating at least one oocyte prior to treatment, isolating at one cumulus cell from each of said isolated oocytes; detecting the levels of expression of at least one gene that is expressed or not expressed at characteristic levels by cumulus cells that are associated with (surround) pregnancy competent oocytes; and assessing the efficacy of said putative fertility treatment based on whether it results in cumulus cells that express at least one pregnancy signature gene at levels more characteristic of cumulus cells that surround pregnancy competent oocytes (than without treatment). As noted, while female human subjects are preferred, the subject methods may be used to assess the efficacy of putative fertility treatments in non-human female animals, e.g., female non-human primates or other suitable animal models for the evaluation of putative human fertility treatments.

Still further, the present invention may be used to enhance the efficacy of in vitro or in vivo fertility treatments. Particularly, oocytes that are found to be “pregnancy incompetent”, or are immature, may be cultured in one or more gene products that are encoded by “pregnancy signature” genes, e.g., hormones, growth factors, differentiation factors, and the like, prior to, during, or after in vivo, or in vitro fertilization. Essentially, these gene products should supplement for a deficiency in nutritional gene products that are ordinarily expressed by cumulus cells that surround “pregnancy competent” oocytes, and which normally nurture oocytes and thereby facilitate the capability of these oocytes to yield viable pregnancies upon fertilization.

Alternatively, one or more gene products encoded by said pregnancy signature genes may be administered to a subject who is discovered not to produce pregnancy competent oocytes according to the methods of the invention. Such administration may be parenteral, e.g., by intravenous, intramuscular, subcutaneous injection or by oral or transdermal administration. Alternatively, these gene products may be administered locally to a target site, e.g., a female ovarian or uterine environment. For example, a female subject may have her uterus or ovary implanted with a drug delivery device that provides for the sustained delivery of one or more gene products encoded by “pregnancy signature” genes.

Thus, in general, the present invention involves the identification and characterization, in terms of gene identity and relative abundance, of genes that are expressed by cumulus cells derived from an egg, preferably human egg, at the time of ovulation, preferably cumulus cells, the expression levels of which correlate to the capability of said egg to give rise to a viable pregnancy upon natural or artificial fertilization and transferal to a suitable uterine environment. Preferably, at least 50 to 100 genes that are significantly upregulated or downregulated, by cumulus cells that correlate to the “pregnancy competency” of an oocyte from which said cumulus cells are associated with will be chosen and monitored in the inventive genetic testing methods.

However, while the invention preferably will select at least 50-100 genes from each of said categories, it is anticipated that the inventive methods alternatively may be practiced by monitoring the expression levels of fewer numbers of cumulus cell expressed genes, wherein said genes are similarly selected to be those which correlate to cumulus cells associated with “pregnancy competent” oocytes, i.e., those that are capable of yielding viable pregnancies.

According to the invention, gene expression levels will preferably be detected by real time RT-PCR. However other known methods, preferably real time detection methods such as mentioned above may be used to detect gene expression. Methods for detecting relative gene expression levels are known in the art and well within the purview of the ordinary skilled artisan.

In the inventive methods, cumulus cells will be isolated from oocytes of different female subjects, the oocytes fertilized by known IVF procedures, and the cumulus cells of the corresponding isolated oocytes being subjected to gene expression analysis, i.e., by isolation of total RNA therefrom, amplification of said total RNA, quantification of the relative gene expression levels of said RNAs by microarray analysis and RT-PCR, and the identification of genes, the expression of which correlates to oocytes that give rise to a viable pregnancy.

To effect such identification, as a separate step, the status of embryos fertilized with oocytes derived from each of said cumulus cell samples will be monitored and pregnancy data recorded. Particularly, the relative birth rate and the health status of the newborn for each oocyte will be recorded and the gene expression levels of cumulus cells associated with each oocyte assessed as a function of pregnancy rate, newborn health, among other parameters, e.g., gender. Based on these results, a set of genes the expression of which correlates to pregnancy/health outcome or gender will be identified. ‘pregnancy signature”)

This set of genes, and the corresponding expression levels is referred to herein as the “pregnancy signature” because these gene expression levels correlate to the development of a viable pregnancy and ultimately the production of a healthy newborn. While this “pregnancy signature” may comprise as many as 50, 100 or even 200 genes, it is anticipated that a fewer number of genes, e.g., on the order of 20 or less genes, may be sufficient to develop a suitable “pregnancy signature”.

The genes which constitute the “pregnancy signature” may include genes which encode gene products that are involved in the nutritional and developmental requirements of the oocyte, i.e., maturation and development, and the potential of the oocyte to be capable of yielding a viable pregnancy. These gene products may include growth factors, hormones, transcription factors, differentiation promoting agents, and the like. After the “pregnancy signature” is obtained, the corresponding genes are sequenced, the DNA sequences are then used to deduce the identify the corresponding polypeptide sequences, and these sequences then compared to databases of available human or other gene sequences to identify the identity of the gene products that correlate to the ability of an oocyte to yield a viable pregnancy. Genes which are differentially expressed by human oocytes are identified infra and contain such pregnancy signature genes. Further statistical analysis of the relative levels of expression of these genes, or subsets of such genes, will identify preferred subsets of these genes that constitute a “pregnancy signature” of a viable oocyte, i.e., one that is pregnancy competent.

As noted previously, these polypeptide gene products deficient in pregnancy incompetent oocytes may be added to in vitro culture media containing oocytes in order to enhance their pregnancy competency or alternatively may be administered in vivo as part of a fertility treatment regimen.

While the invention has been described in detail, the following example is provided to further illustrate the invention and its preferred embodiments.

### EXAMPLE

Description

Phase I: At the clinic, embryologists will remove the cumulus cells of two eggs and fertilize them. Embryos will be transferred to the uterus of a woman and cumulus cells sent to the laboratory for analysis. Once the cells arrive to the laboratory, RNA will be isolated and microarray analysis performed using Affymetrix platform. Pregnancy tests will be done by ultrasound on day 30 and embryonic sacs counted. There will be three kinds of outcomes: 1) 0 sacs; 2) 1 sac and 3) 2 sacs. A minimum of 30 volunteer women will participate during this phase. Ten with no sacs, ten with one sac and ten with 2 sacs. Pregnancy data will be correlated with gene expression obtained from the cumulus cells isolated from those same eggs. One hundred genes that directly correlate with pregnancy—either by upregulation or downregulation—will be further analyzed using real time RT-PCR. The best 20 genes that correlate with pregnancy (positively or negatively) will be called “pregnancy signature” and used for later testing at the clinic.

Phase II: Blind validation of genes in the pregnancy signature. At the clinic, the embryologist will isolate RNA from cumulus cells from each oocyte that will be later fertilized. Half of the RNA will be sent to our laboratory and the rest will be used for real time RT-PCR analysis to be performed on site. Gene expression of the “pregnancy signature” will be measured. Embryologists will transfer embryos without knowing the outcome of gene expression analysis. One hundred women will be asked to participate as volunteers in this part of the study. At the time pregnancy results are obtained, the study will be unmasked and results from each individual will be correlated with gene expression analysis. We anticipate that the “pregnancy signature” put forward in phase 1 will be validated during this phase.

Alternative strategy: In the event of an unexpected outcome i.e., the pregnancy signature is not validated; microarray analysis will be run once more using the RNA provided by the clinic in phase 2. It is anticipated that having 100 more samples will result in the identification of a clear pattern of gene expression in cumulus cells from eggs capable (or non-capable) of generating a healthy pregnancy/baby.

Using microarray analysis as described above, the genes identified infra were found to be differentially expressed by cumulus cells obtained from eggs of women donors. The expression of those particular genes which correlate to pregnancy (positive or negative) will establish a “pregnancy signature”, i.e., genes the expression or absence of expression of which correlates to a positive pregnancy outcome and “infertility signature”, i.e., specific genes the expression or absence of expression correlate to fertility problems or abnormalities.

This is effected preferably by microarray analysis. For example, comparison of expression between two samples on filter arrays may be performed by comparing nucleic acids obtained from normal oocyte cells to those obtained from a donor suspected of having ovarian dysfunction that renders oocytes pregnancy incompetent on two duplicate filters or alternatively a single filter may be used that is stripped and hybridized sequentially.

Direct comparison of gene expression in two samples can be achieved on glass arrays by labeling the two samples with different flourophores. This technique allows the evolution of repression of gene expression as well as induction of expression. The two flouresently-labeled cDNAs are then mixed and hybridized on a single glass or filter array. Glass arrays have the advantage of allowing the simultaneous analysis of two samples on the same array under the same hybridization conditions.

Gene arrays containing sequences of genes implicated in pregnancy (“pregnancy signature”) will allow high-throughput screening of individuals for diagnostic purposes or tailor-made treatments.

Arrays of polynucleotides, the expression of which corresponds to, or are complementary to the sequences of genes identified by the method of the invention therefore provide a further aspect of the invention. Such an array will include at least two nucleic acid sequences, preferably at least 10, and more preferably at least 20, e.g., 50 genes or more that correspond to the sequence of, or are complementary to genes, the expression of which (positive or negative) the positive pregnancy outcome in cells obtained from oocyte donors, e.g., women suspected to have ovarian dysfunction as a result of disease, age, and the like. Protein arrays form a further aspect of the invention and will contain polypeptides encoded by such pregnancy signature genes or antibodies which bind thereto.

Recent developments in the field of protein and antibody arrays allow the simultaneous detection of a large number of proteins.

1. A method of identifying oocytes that are capable of giving rise to a viable pregnancy when fertilized comprising the following steps:
(i) obtaining at least one cell that is associated with an oocyte in vivo;
(ii) assaying the expression of at least one gene by said cell associated with an oocyte, the expression of which correlates to the capability of an oocyte associated with said cell to yield a viable pregnancy upon fertilization and transferal into a suitable uterine environment; and
(iii) identifying, based on the level of expression of said at least one gene, whether said oocytes is potentially capable of yielding a viable pregnancy upon fertilization and transferal into a suitable uterine environment.

2. The method of claim 1, wherein said oocyte is a mammalian oocyte.

3. The method of claim 2, wherein said oocyte is a human oocyte.

4. The method of claim 2, wherein said ooycte is a non-human primate oocyte.

5. The method of claim 1 wherein said oocyte-associated cell is a cumulus cell.

6. The method of claim 1, wherein the expression of at least 5 genes, the expression of which correlates to the capability of an oocyte to potentially yield a viable pregnancy are identified.

7. The method of claim 6, wherein the expression of at least 10 genes, the expression of which correlates to the capability of an oocyte to potentially yield a viable pregnancy are measured.

8. The method of claim 7, wherein the expression of at least 15 genes, the expression of which correlates to the capability of an oocyte to potentially yield a viable pregnancy are identified.

9. The method of claim 8, wherein the expression of at least 20 genes, the expression of which correlates to the capability of an oocyte to potentially yield a viable pregnancy are identified.

10. The method of claim 9, wherein the expression of at least 20 to 50 genes, the expression of which correlates to the capability of an oocyte to potentially yield a viable pregnancy are identified.

11. The method of claim 10, wherein the expression of at least 50 to 100 genes, the expression of which correlates to the capability of an oocyte to potentially yield a viable pregnancy are identified.

12. The method of claim 1 wherein the method of assaying gene expression uses a method that monitors differential gene expression.

13. The method of claim 12 wherein said method comprises indexing differential display reverse transcriptase polymerase chain reaction (DDRT-PCR)

14. The method of claim 3, wherein the oocyte is obtained from a woman who is at least 25 years old.

15. The method of claim 14, wherein the oocyte is obtained from a woman who is at least 30 years old.

16. The method of claim 15, wherein the oocyte is obtained from a woman who is at least 35 years old.

17. The method of claim 16, wherein the oocyte is obtained from a woman who is at least 40 years old.

18. The method of claim 3, wherein the aberrant expression of said at least one gene by said oocyte-associated cell is correlated to a condition selected from menopause, cancer, ovarian dysfunction, ovarian cyst, autoimmune disorder and hormonal dysfunction.

19. A method of assessing the efficacy of a fertility treatment comprising:
(i) treating a woman with a putative fertility enhancing treatment;
(ii) obtaining an oocyte and cells associated therewith from said woman after treatment and measuring the expression of at least one gene by a cell associated with said oocyte, the expression of which positively or negatively correlates to the capability of said oocyte to yield a viable pregnancy upon fertilization and transferal to a suitable uterine environment; and
(iii) evaluating whether said treatment is effective based on the level of expression of said at least one gene by said oocyte-associated cell.

20. The method of claim 19, wherein said fertility treatment comprises hormonal therapy.

21. The method of claim 20, wherein the subject is menopausal and the treatment comprises hormone replacement therapy.

22. The method of claim 1, wherein gene expression is detected by real-time polymerase chain reaction (RT-PCR).

23. The method of claim 19, wherein gene expression is detected by RT-PCR.

24. The method of claim 19 wherein gene expression is detected differentially by indexing differential display reverse transcriptase polymerase chain reaction (DDRT-PCR).

25. The method of claim 19 wherein said oocyte-associated cell is a cumulus cell.

26. A method of evaluating fertility potential in a subject comprising comparing the expression levels of specific genes (‘pregnancy signature genes”) in a cell associated with an oocyte, wherein said genes are expressed at characteristic levels (upregulated or downregulated) in an oocyte-associated cell, wherein the oocyte associated with cell is capable of yielding a viable pregnancy; and determining whether said subject is potentially “pregnancy competent” based on whether a cell associated with an oocyte of said subject expresses one or more pregnancy signature genes at levels characteristic of pregnancy competent oocytes.

27. The method of claim 26 wherein said oocyte-associated cell is a cumulus cell.

28. The method of claim 26 wherein said method monitors differential gene expression by indexing differential display reverse transcriptase polymerase chain reaction (DDRT-PCR).

29. The method of claim 1 wherein gene expression is detected using antibodies that specifically bind to “pregnancy signature” polypeptides.

30. An array of at least two different polynucleotide sequences or polypeptides encoded thereby the levels of expression or absence of expression correlate to the pregnancy potential of a cell that is associated with a mammalian oocyte expressing said polynucleotide sequences or polypeptides.

31. The array of claim 30 which is a polynucleotide sequence array.

32. The array of claim 30 which is a polypeptide array.

33. The array of claim 31 which comprises at least 10 polynucleotide sequences.

34. The array of claim 32 which comprises at least 10 polypeptides.

35. A method of using an array according to claim 30 to detect whether a cell expresses pregnancy signature genes.

36. A method of using an array according to claim 30 to determine the efficiency of a fertility treatment by detecting whether cells obtained from a fertility treatment subject express one or more nucleic acid or polypeptides contained in said array.

37. The array of claim 30 which is comprised on a filter or glass chip.

38. The array of claim 37 which comprised a detectable label.

39. The array of claim 38 wherein said label is a fluorophore.
